(19) United States
(12) Patent Application Publication
(10) Pub. No.: US 2021/0331000 A1
(43) Pub. Date: Oct. 28, 2021

(54) HYGIENIC FACE MASK

(71) Applicant: Inland Concrete Products, Inc., Fontana, CA (US)

(72) Inventors: John H. Gilner, Santa Barbara, CA (US); G. Douglas Hartzheim, Bozeman, MT (US); Steven R. Wilbur, Yucaipa, CA (US)

(73) Assignee: Inland Concrete Products, Inc., Fontana, CA (US)

(21) Appl. No.: 16/994,350

(22) Filed: Aug. 14, 2020

Related U.S. Application Data
(63) Continuation-in-part of application No. 29/732,485, filed on Apr. 24, 2020, now Pat. No. D923775.

(51) Int. Cl. A62B 18/02; A62B 23/02; A62B 18/08

(52) U.S. Cl. CPC A62B 18/025; A62B 18/086; A62B 23/02

(57) ABSTRACT

A mask assembly includes a mask body configured to cover a wearer's nose and mouth. The mask body has a breathing area with breathing openings and a straw port. A replaceable filter medium is configured to cover the breathing openings on the interior of the mask body when installed thereon in a removable filter medium retainer element. A removable face plate overlies, and is spaced from, the breathing area on the exterior of the mask, so as to allow air to flow through the breathing openings and around the sides of the face plate, while blocking particulate matter from passage to or from the nose or mouth of the wearer. The face plate, the filter medium retainer element, and the filter medium may have openings that are alignable with the straw port.

### CROSS-REFERENCE TO RELATED APPLICATIONS

This application is a continuation-in-part of U.S. Design patent application No. 29/732,485, filed Apr. 24, 2020, which is incorporated by reference herein.

### FEDERAL SPONSORED RESEARCH OR DEVELOPMENT

Not applicable

### BACKGROUND

The present disclosure pertains to hygienic face masks used to filter particulate matter and/or aerosol droplets. More particularly, it relates to face masks that are suitable for use in public areas and that may be easily reconfigured for various functionalities.

Face masks have long been used to allow people to enter areas having airborne particulate matter while minimizing the amount of particulate matter inhaled by a person wearing a face mask. Face masks have also been used hygienically for reducing or minimizing inhalation and exhalation of aerosolized droplets that may contain infectious pathogens. Traditionally, masks use filters that catch particulate matter that could be carried to and from the nose and mouth of a wearer. Masks may also be used in conjunction with other physical barriers, such as face shields or barriers of transparent plastic or glass, to minimize particulate matter that could contact the mask. However, such physical barriers are oftentimes inconvenient to use in conjunction with a mask, often requiring a separate physical apparatus to be erected around the mask wearer, or a separate apparatus that couples a face shield to the head of the wearer.

Wearing a face mask can also limit the activities a face mask wearer can perform. For example, a person wearing a typical hygienic face mask is unable to eat food, drink from a straw, or wear an oxygen nasal cannula, as they would be able to do without wearing the face mask. This drawback has led to a reluctance on the part of many people to wear hygienic masks when in public, which is contrary to best practices in controlling the spread of infectious diseases.

Thus, it is desired to provide a hygienic face mask that provides greater ease and convenience of use without compromising the hygienic functions of the facial mask.

### SUMMARY

The present disclosure relates to a hygienic mask assembly including a mask body with a breathing area provided with a plurality of breathing openings, and face plate removably attachable to the mask body so as to be displaced from the breathing openings in order to obstruct the path of particulate matter, such as aerosol droplets, through the breathing openings, while permitting air to flow into and out of the breathing openings. The mask body accommodates a detachable filter retainer element in its interior surface that is configured to hold a replaceable filter medium against the interior surface of the mask body. In accordance with aspects of this disclosure, the mask body may also have a plurality of ports that allow a wearer to interact with external devices, such as straws, sipping tubes, and oxygen tubes.

More specifically, a mask assembly in accordance with an aspect of this disclosure includes a mask body configured to cover a nose and mouth of a wearer. The mask body defines an exterior surface and an interior surface, and it has a breathing area having breathing openings to permit passage of air between the exterior and the interior surfaces. A face plate may be removably attachable to the front surface of the mask body, so that, when so attached, it is sized and configured to cover a line of sight of each of the breathing openings of the mask body, with a rear surface that is spaced from the breathing openings of the mask body to allow passage of air through the breathing openings. A filter medium may be removably installed on the interior surface of the mask body to cover the breathing openings.

In accordance with an aspect of this disclosure, the mask body may have a straw port in the breathing area. The straw port may comprise a straw-pierceable element surrounded by a reinforcement collar. The face plate advantageously has an opening that aligns with the straw port when the face plate is attached to the exterior surface of the mask body.

In accordance with an aspect of this disclosure, a filter retainer element may be removably installed in the interior of the mask body to hold the filter medium in place against the interior surface of the mask body so as to cover breathing openings. The filter retainer element may include a filter retainer opening, and the filter medium may include a filter opening, wherein the filter retainer opening, the filter opening, the face plate opening, and the straw port are each aligned with one another when the filter retainer element and the filter medium are installed in the interior surface of the mask body.

In accordance with an aspect of this disclosure, the mask body may have a pair of upper eyelets and a pair of lower eyelets. The pair of upper eyelets and the pair of lower eyelets may extend from opposing sides of the mask body, where the pair of upper eyelets are disposed or to be above the pair of lower eyelets when the mask body is worn on the face of the wearer. By coupling a head strap to the eyelets in various configurations, the mask body could be tightened against the face of the wearer along different edges of the mask body, as explained further below.

### BRIEF DESCRIPTION OF THE DRAWINGS

FIG. 1A is a front elevation view of an embodiment of a mask body in accordance with an aspect of this disclosure.

FIG. 1B is a rear elevation view of the mask body of FIG. 1A.

FIGS. 1C and 1D are rear perspective views of the mask body of FIG. 1A.

FIG. 2 is a front elevation view of the mask body of FIGS. 1A-1D showing two alternative strap configurations.

FIG. 3A is a front perspective view of an embodiment of a face plate for removable attachment to the mask body of FIGS. 1A-1D.

FIG. 3B is a rear perspective view of the face plate of FIG. 3A.

FIG. 3C is a front perspective view of the face plate of FIG. 3A having a first indicia label on the front surface.

FIG. 3D is a front perspective view of the face plate of FIG. 3A having a second indicia label on the front surface.

FIG. 4 is a front elevation view of an embodiment of a filter medium for use with the mask body of FIG. 1A.

FIG. 5A is a front perspective view of an embodiment of a filter retainer element for use with the mask body of FIG. 1A.

FIG. 5B is a rear perspective view of the filter retainer element of FIG. 5A.

FIG. 6A is a front elevation view of a mask assembly comprising the mask body of FIG. 1A with a face plate in accordance with FIGS. 3A and 3B attached thereto, in accordance with an aspect of the disclosure.

FIG. 6B is a rear elevation view of the mask assembly of FIG. 6A, with a filter retainer element in accordance with FIGS. 5A and 5B attached thereto, in accordance with an aspect of the disclosure.

FIG. 6C is a bottom perspective view of the mask assembly of FIG. 6A.

FIG. 7 is a top perspective exploded view of the mask assembly of FIG. 6A.

FIG. 8 is an exploded perspective view of a mask assembly comprising a face plate, a mask body, a filter medium, and a filter retainer, in accordance with aspects of the disclosure.

FIG. 9 is a side elevation view of an exemplary embodiment of a plug configured for use with the mask assembly of FIG. 6A.

FIG. 10 is a perspective view of an alternative embodiment of a mask assembly in accordance with aspects of this disclosure.

### DETAILED DESCRIPTION

A mask assembly in accordance with an aspect of this disclosure includes a mask body that is configured to cover a nose and mouth of a wearer. The mask body has a breathing area that has breathing openings between an exterior surface of the mask and an interior surface of the mask, allowing for air to pass between the exterior surface and the interior surface via the breathing openings. The breathing openings may be of any suitable shape and size, for example, a series of ovoid or rectangular holes comprising a grille, or a series of circular holes comprising a sieve. The breathing area is in front of a mouth of a wearer of the mask when the mask is worn, thereby allowing the wearer to exhale and inhale through the breathing openings. However, this configuration also provides a direct path for exhaled droplets to exit the mask body at a high velocity via the breathing openings, and for inhaled droplets to enter the nose and mouth of the wearer via the breathing openings.

In accordance with one aspect of the disclosure, a face plate may be removably coupled or attached to the mask body to cover some or all the breathing openings of the mask body. As used herein, a face plate that “covers” a breathing opening blocks a more or less linear path from the breathing opening. In other words, the direct flight path of droplets to or from the wearer is blocked by the face plate. In one aspect of the disclosure, when the face plate is attached to the mask body, most or all of the breathing openings are obscured from a frontal view by the face plate. In another aspect, the face plate may cover the entire breathing area, such that the face plate has a width greater than the width of the breathing area and a height greater than the height of the breathing area.

The rear surface of the face plate is advantageously spaced from the breathing openings of the mask body to allow passage of air through the breathing openings around the edges of the face plate. Any suitable mechanism could be utilized to space the face plate from the front of the mask body about the breathing area, such as by coupling the face plate to the mask body using one or more elongated supports, and/or by disposing a plurality of standoffs on the rear surface of the face plate to ensure that the rear surface of the face plate does not block air passage in and out of the breathing openings.

In accordance with an aspect of the disclosure, the mask body may include one or more ports that allow a fluid conduit to pass between an exterior surface of the mask and an interior surface of the mask via a port opening. Such conduits may include, for example, a drinking straw or a sipping tube, or an oxygen line or cannula. The material around an open port may comprise an elastic material that elastically compresses around the conduit to provide a substantially airtight seal around the conduit, ensuring that the filtration function of the mask is maintained as much as possible. Such ports could be positioned in any suitable location along the surface of the mask body, such as in the breathing area between one or more breathing openings, or along a side wall of the mask body. The port opening could be reinforced with a collar that surrounds the port opening and extends from a surface of the mask body, such as at least 3 mm or 5 mm. In one aspect of the disclosure, one or more ports could be manufactured to be a pierceable element that is thinner than the material of the mask body as a whole. For example, a pierceable element could have a maximum thickness of about 1.0 to about 1.5 mm, in a mask body having a thickness of between about 1.5 mm and 2.0 mm, although these dimensions are exemplary only. The pierceable element could also be perforated or scored to provide a visible or tangible piercing guide, such as a scored cross or a recessed point, on at least one side of the mask body.

The mask body may be held in place on a face of the wearer in any suitable manner, for example by using one or more elastic straps that are connected to the mask body. In some embodiments, the exterior surface of the mask may have one or more eyelets through which one or more straps are passed through. The eyelets may be arranged along an edge or a perimeter of the mask, for example at several (e.g., four) points along a perimeter of the mask, which would allow for two straps to be used to hold the mask body in place against the face of the wearer by passing the straps through the eyelets of the mask.

The mask body may be made of any suitable rigid, semi-rigid, or even flexible material, such as, for example, a thermoplastic elastomer (TPE) or a polycarbonate plastic, or it may comprise a mixture of or a combination of different materials. The mask body may be injection molded, thermo-formed, 3-D printed, or assembled using discrete parts. In some aspects, portions of the mask body could be different thicknesses to provide areas of different degrees of flexibility, depending upon need. For example, a flange of the mask body may have a smaller thickness to provide more flexibility for the sake of comfort and fit, while the nose-covering portion may have a larger thickness to provide more rigidity.

In accordance with one aspect of the disclosure, a replaceable filter medium is removably installed the mask body to filter air passing through the breathing openings of the mask. The filter medium may be sized and configured to cover all or most of the breathing openings located in the breathing area of the mask in one aspect, while in another aspect a plurality of breathing filters could be used to filter different portions of the breathing area. Such configurations allow the filter medium to filter air that passes through the breathing openings in either direction. The filter medium may comprise any suitable material or materials that filter particulate matter and aerosol droplets from air passing through the breathing medium. Depending on the application and need, the filter medium may be or include filter paper or cloth, and in some case may include activated carbon. The filter medium may be removably secured to the interior surface of the breathing area of the mask body in any suitable manner, such as by using a filter retainer element that detachably couples to the interior surface of the mask body to hold the filter medium in place against the breathing area of the mask body, or, for example, by providing a series of projections that extend from a surface of the mask body that pierce through portions of the filter medium to hold the filter medium in place.

A filter retainer element configured to hold the filter medium in place on the mask body could be configured to be removably coupled to the mask body to enable a user to change the filter medium from time to time. The filter retainer element may be configured to be removably installed in the mask body such that the filter medium is held against at least some of the breathing openings of the mask by the filter retainer element. The filter retainer element may be installed in the mask body in any suitable manner, such as by mating with matching indents or detents, or by having an attachment mechanism that slides into matching projections or recesses in the mask body.

In accordance with one aspect of the disclosure, the face plate could have one or more indicia labels displayed on its front surface. Such indicia labels could be permanently or removably applied to the front of the face plate in any suitable manner, such as an adhesive or thermal bond, or they may be molded into, or otherwise formed in, the front surface of the face plate. A plurality of interchangeable face plates could be provided in a mask kit, allowing a user to change the displayed indicia label by switching one face plate with another. The face plate could comprise any suitable material or combination of materials, preferably a plastic, such as polypropylene.

In accordance with some aspects of the disclosure, any of the elements of the mask apparatus, such as the filter medium, the filter retainer element, and/or the face plate, could have openings that align with one or more ports of the mask body to allow a fluid conduit, such as a straw, to extend through the openings and be accessible from within the mask body. For example, a straw could be extended through an opening in the face plate, then through a straw port in the mask body, then through an opening in a filter medium and finally through an opening in a filter retainer element to reach the mouth of the wearer of the face mask. In such embodiments, the material about an opening of at least two of the elements could comprise a rigid material to help support the conduit when it is installed through a port in the mask body. For example, the face plate and the filter retainer element could be made of a rigid polypropylene to help support a straw and reduce damage to a less rigid TPE material comprising the material around the port of the mask body.

Contemplated ports could be disposed in areas outside of the breathing area of the mask body, such as the sides, bottom, or top walls of the mask body as well. A plurality of ports may also be placed on opposing sides of the mask body to allow a conduit to be extended into the mask body via one port and out of the mask body via another port. For example, an oxygen tube could be installed through one port to deliver oxygen to a nasal cannula on one side of the mask, and another oxygen tube could be extended through another port to deliver oxygen to the nasal cannula on another side of the mask. In other aspects, multiple ports could be provided in a mask body to provide similar functions, such as a sipping tube port located outside a breathing area of the mask body and a straw port located within the breathing area of the mask body. Where a mask body has a plurality of ports, the ports are preferably sealed, but pierceable, to allow a user to create an opening at any sealed port location by penetrating the mask body with a rigid object, such as a straw, awl, or knife.

Contemplated masks could be provided in a mask assembly or kit comprising a mask body, a filter retainer element, one or more filter media, and one or more face plates. The filter media could be changed as they are used, for example between days that the mask is used or between events that the mask is used. Face plates could be interchangeable with other face plates to provide different visual indicia, such as team logos or business slogans. Since some users may not wish to use a port at all times, refills for the filter medium could be provided with or without openings that align with a port, and one or more removable closures could be provided to close a port when it is not in use.

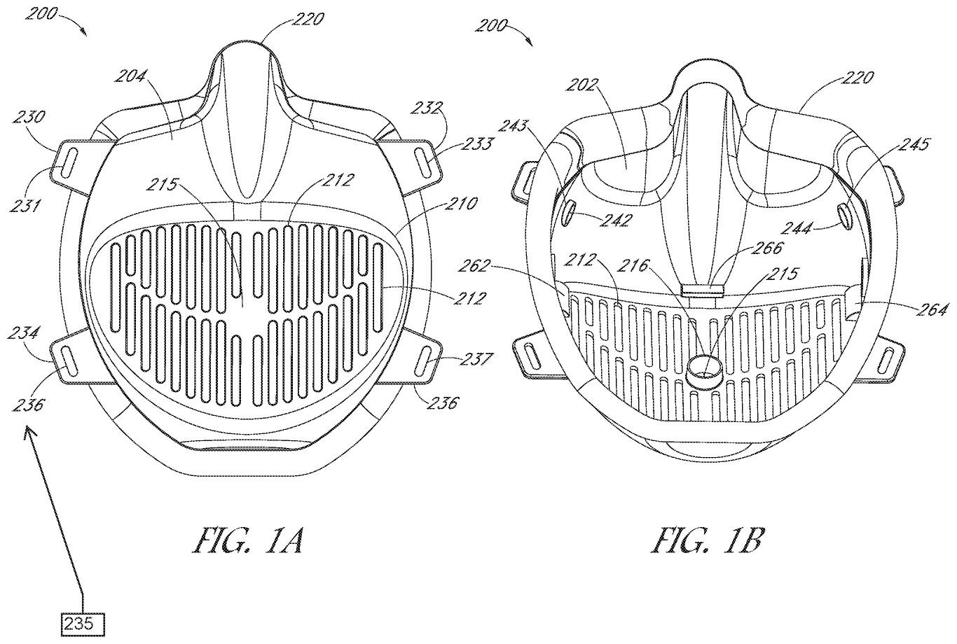

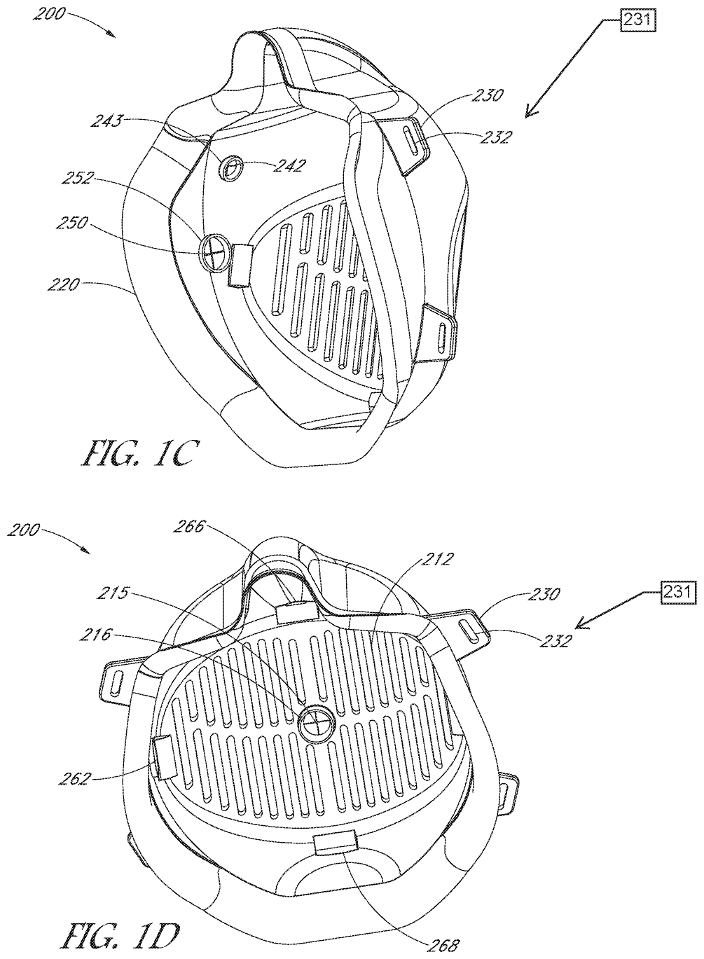

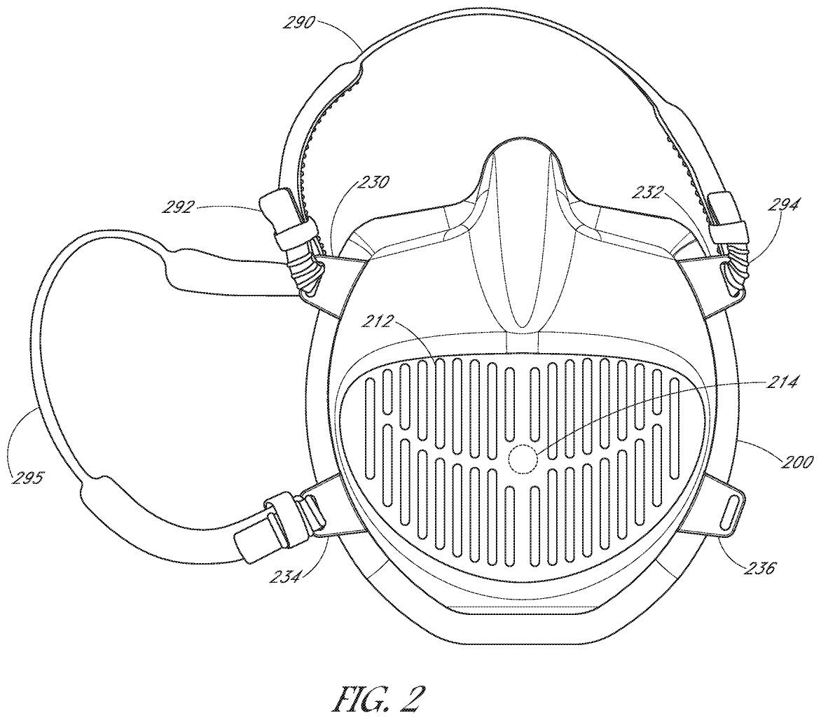

FIGS. 1A-1D illustrate a mask shell or body 200 defining an interior surface 202 and an exterior surface 204. The mask body 200 includes a breathing area 210 in a portion that would cover the mouth and, advantageously, the nostrils of a wearer (not shown). The breathing area 210 has an array or grid comprising a plurality of breathing openings 212 that allow air to pass bidirectionally through the mask body 200 to and from the wearer as the wearer inhales and exhales, respectively. While the breathing openings 212 are shown as a series of parallel, vertically aligned, elongate apertures, the breathing openings 212 could be disposed and arranged in any suitable manner, such as horizontally aligned elongate apertures, circular holes scattered throughout the breathing area 210, or a checkered/grille pattern.

The mask body 200 has a plurality of ports configured to allow any of a variety of tubular devices or fluid conduits to be passed through the mask body 200, depending upon need. For example, one or more oxygen ports may be provided in the mask body 200, such as, for example, a first oxygen port 242 on the left side of the mask body, and/or a second oxygen port 244 on the right side of the mask body 200. The one or more oxygen ports are dimensioned to allow an oxygen line or tube 910 and/or 920 (see FIG. 10) to be extended through the mask body and deliver oxygen to the wearer, advantageously through a nasal cannula (not shown) or other breathing apparatus. A straw port 215 may advantageously be provided at or near the center of the breathing area 210 to allow a straw 500 (see FIGS. 8 and 10) to be extended to the wearer through the breathing area 210 of the mask body 200. A sipping tube port 250 may also be provided at a suitable location in the mask body 200 to allow a sipping tube 600 (see FIGS. 8 and 10) to be extended through the mask body 200 to the wearer. It will be readily understood that the number and location of the ports are matters of design choice, depending on the particular needs or desires of the wearer, and thus may be varied accordingly.

Each of the ports 242, 244, 215, and 250 may be defined by an area of reduced material thickness, such that it is pierceable either by a tubular device or conduit itself, or by a separate piercing implement (not shown). Each pierceable port area may have a smooth surface that is continuous with the exterior surface 204 of the mask body 200, and it may have a scored surface on the interior surface 202 of the mask body 200 marking the pierceable port area. As used herein, a “pierceable” area is an area that has a thickness that is less than the thickness of the surrounding mask body material, whereby the pierceable area may be breached or penetrated by the conduit or implement without undue force, such as, for example, less than about 10 Newtons. Alternatively, or in addition, the pierceable area may be defined by score lines that facilitate its separation from the body of the mask, and/or it may be made of a different, weaker material.

In some aspects, the material comprising the pierceable area of the port may comprise an elastomeric material, such as a TPE material, which creates a seal around any device that is inserted into the port when the elastomeric material restores itself towards its original, non-stretched configuration. In some aspects, the pierceable area of the port may comprise a small hole that elastically expands when a device is inserted into it and elastically contracts to a nearly or partially closed configuration when the device is removed from it.

The pierceable areas of some or all the ports may be surrounded by an annular collar on the interior mask body surface 202 to provide reinforcement for the pierceable area and to enhance the support for a device that is inserted through a port opening. For example, the straw port 215 may have a collar 216, the sipping tube port 250 may have a collar 252, the left oxygen port 242 may have a collar 243, and the right oxygen port 244 may have a collar 245. The collars 216, 252, 243, and 245 (if present) preferably extend from about 2-5 mm from the interior surface 202 of the mask body 200, and they may have a width/thickness of about 2-3 mm. While each of the collars is shown herein as extending from the interior surface 202 of the mask body 200, each collar could alternatively or additionally extend from the exterior surface 204 of the mask body 200 in other aspects. In some aspects, a port may not have a collar that extends from either the interior surface 202 or the exterior surface 204 of the mask body 200.

The mask body 200 may have a flange 220 that is shaped to surround the nose and mouth of the wearer when the mask body 200 is worn. The flange 220 is shaped to abut the face of a wearer and provide a substantially, but not necessarily fully, airtight seal. Such a seal obstructs air from traveling between the interior surface 202 and the exterior surface 204 via the perimeter of the mask body 200. By forming such a seal between the flange 220 and the face of the wearer, the vast majority of the inhaled airflow to the wearer, and exhaled airflow from the wearer, must pass through the breathing openings 212. The flange 220 is also preferably made of an elastomeric material that conforms to the face of the wearer to improve the fit of the seal between the flange 220 and the face of the wearer.

FIG. 2 illustrates a mask 200 having two alternative strap configurations. The exterior surface 204 of mask body 200 has a pair of upper eyelets 230, 232, and a pair of lower eyelets 234, 236 extending from opposite sides of the mask body for securing one or more straps to hold the mask body 200 to the head of the wearer. Each eyelet 230, 232, 234, and 236 comprises an aperture 231, 233, 235, and 237, respectively, allowing each of the eyelets to be used to secure one end of a strap, such as a head strap 290 and a pair of ear straps 295 (only one of which is shown). Specifically, each end of the head strap 290 may be secured to one of the upper eyelets 230, 232 so as to wrap around the back of the wearer's head. Each end of a first ear strap 295 may be secured to one of the upper eyelets and one of the lower eyelets on one side of the mask body, so as to wrap around an ear of the wearer. Thus, the head strap 290 is shown as a common projection strap having a first end 292 and a second end 294 that are respectively securable, by conventional means, to the upper eyelets 230, 232 through the apertures 231, 233 respectively. In cases where a wearer wishes to wear the straps horizontally about the head of the wearer, the head strap 290 would be coupled to the upper eyelets 230, 232, while a second head strap, would be coupled to the lower eyelets 234, 236. If the wearer wishes to wear the straps vertically about the ears of the wearer, the head strap 290 would be omitted, and a first ear strap 295 would be coupled to the eyelets 230 and 234 on one side of the mask body, while a second ear strap 295 would be coupled to the eyelets 232 and 236 on the other side of the mask body. Straps could be made of any suitable material, either elastic or non-elastic, as preferred by the wearer.

In some aspects, the flange 220 of the mask body 200 may be configured to form an airtight or substantially airtight seal about the mouth and nose of the wearer—particularly above the nose of the wearer. Such a seal may serve to obstruct exhaled air from fogging up glasses that may be worn by the wearer. In addition, by configuring the mask 200 to use differing strap configurations, the mask 200 can provide various advantages relative to masks configured to use only a single strap configuration. For example, straps attached horizontally to the upper eyelets 230 and 232, and to the lower eyelets 234 and 236, respectively, would provide a snugger fit of the flange 220 against the face of the wearer, thereby helping to obstruct air flowing past the flange 220 of the mask body 200. In effect, the airtight seal located above the nose of the wearer would be tightened by the strap coupled to the upper eyelets 230 and 232, which reduces the tendency of exhaled air to fog up a wearer's glasses. Straps attached vertically to the left eyelets 232 and 236, and to the right eyelets 230 and 234, respectively, would sit more comfortably around the ears of a wearer, and could allow for the mask 200 to be more quickly removed and placed back onto the wearer's face when desired.

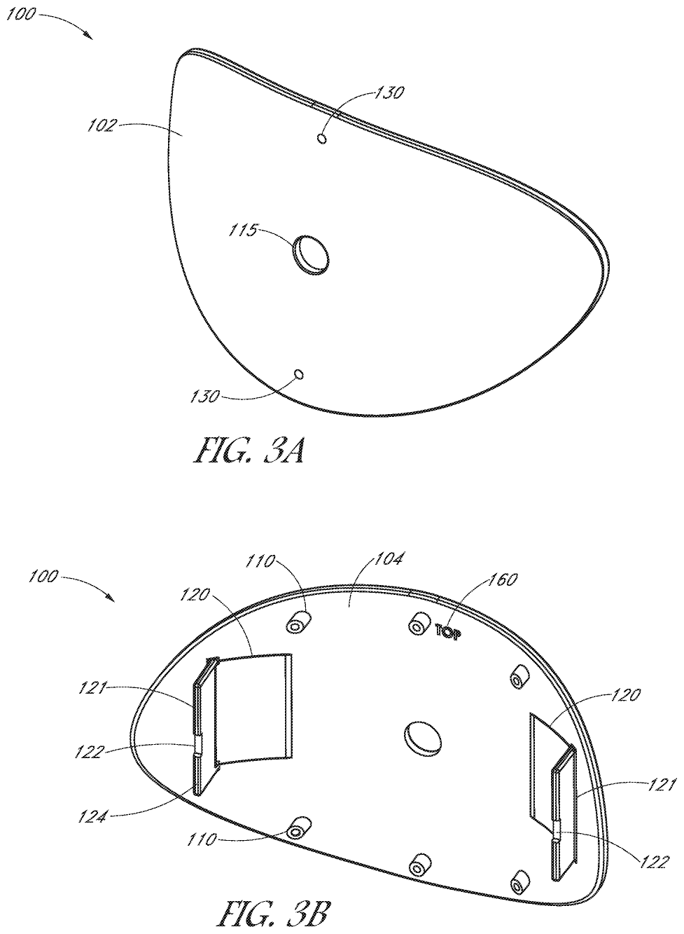

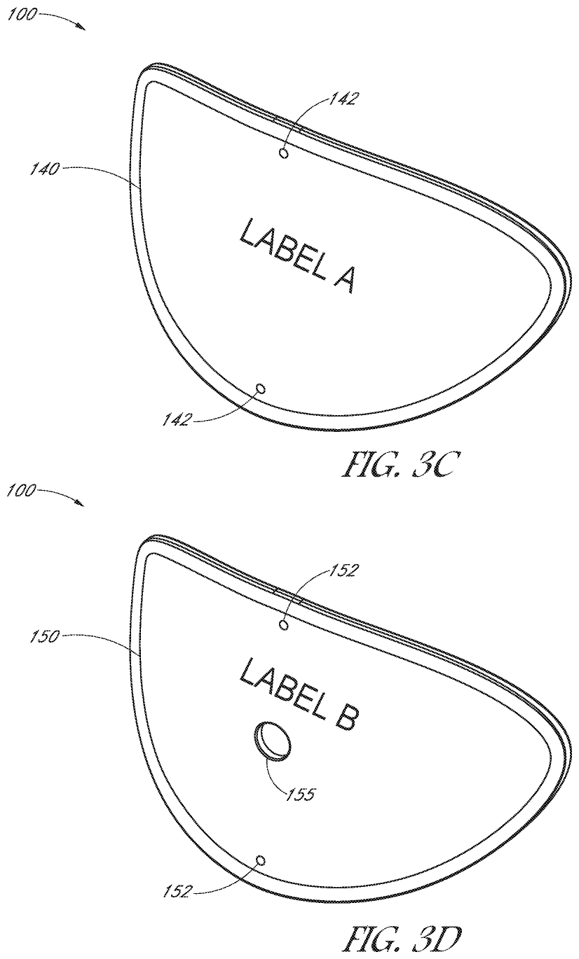

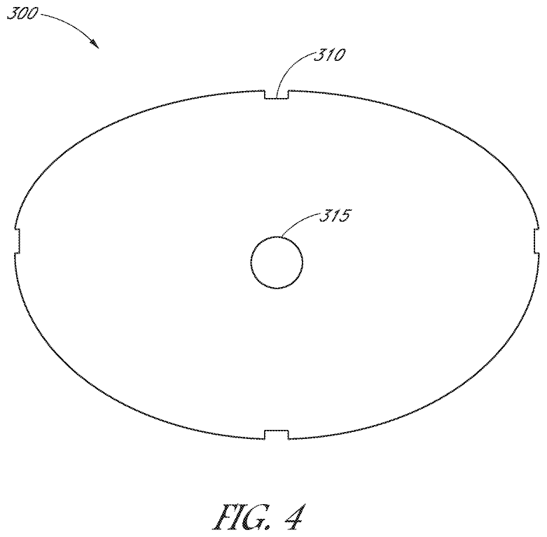

FIGS. 3A-3D illustrate a detachable face plate 100 that may be removably attachable to the mask body 200 in accordance with aspects of the disclosure. The face plate 100 has a front surface 102 that, when the face plate 100 is attached to the mask body, faces away from the mask body, and a rear surface 104 that faces towards the mask body 200 when the face plate 100 is attached. The face plate 100 is dimensioned to correspond roughly to the breathing area 210 of the mask body, so that when it is attached (as described below), it approximately covers the breathing area, albeit spaced distally (from the viewpoint of the wearer) from it. The face plate 100 advantageously has a central opening 115 that is aligned with the straw port 215 of the mask body 200 when the face plate is attached, to allow a straw to pass through the aligned central opening 115 and the straw port 215.

The front surface 102 of the face plate 100 may advantageously have two or more guides 130 that could be used to align an indicia label applied to the front surface 102, such as a first indicia label 140 shown in FIG. 3C or a second indicia label 150 shown in FIG. 3D. The indicia labels 140, 150 may have visual or tactile features, such as holes 142 in the first indicia label 140 or holes 152 in the second indicia label 150, that align with the guides 130 when applying the indicia label to the front surface 102 of face plate 100. Other features could alternatively be used as alignment mechanisms, such as transparent/translucent areas with visual features, such as circles, lines, or other shapes.

An indicia label could be applied to or fixed to the front surface 102 of the face plate 100 in any suitable manner. Preferably, the indicia label 140, 150 is in the form of a sheet of material, such as a decal, that may be affixed adhesively or thermally, either permanently or removably. Alternatively, the face plate 100 may be made of a material that has a static affection for an indicia label, such as a smooth plastic material that has a static affection for a vinyl or a polyester indicia label. The first indicia label 140 in FIG. 3C does not have an opening that matches the central opening 115 of the face plate 100, whereas the second indicia label 150 of FIG. 3D has an opening 155 that matches the central opening 115 of the face plate 100. Thus, either the first indicia label 140 or the second indicia label 150 may be used, depending on whether the wearer wants to use the straw port 215 of the mask body. In some aspects, an indicia label may be weakened or perforated around the area of the central opening 115 (when applied to the face plate 100) to allow a user to pierce the indicia label in the area of the central opening 115 to use the straw port 215.

The face plate 100 may include alignment indicia 160 printed or embossed on a surface of the face plate 100 to provide a visual guide to a user to properly align the face plate 100 when removably attaching the face plate 100 to the mask body 200.

Referring to FIG. 3B, the face plate 100 may include one or more (preferably two, as shown) mounting elements 120 on its rear (proximal) surface 104 that are configured to detachably mount the face plate 100 to the front surface of the mask body 200. In the illustrated embodiment, there are two mounting elements 120, one near each of the opposite side edges of the face plate 100. Each of the mounting elements 120 in the illustrated exemplary embodiment includes a vertical, proximally-extending panel 121 terminating in a hook portion 124 by which the face plate 100 is removably attachable to the mask body 200, as explained below. The face plate 100 preferably comprises a flexible material that can flex to distort out of its initial shape and yet spring back into its original shape to hold face plate 100 in place on the front of the mask body distally from the breathing area 210. Thus, when attaching the face plate 100 to the front of the mask body 200 in the manner described below, a user would manipulate the face plate 100 to engage the hook portions 124 with appropriate apertures or openings (as described below) in the mask body.

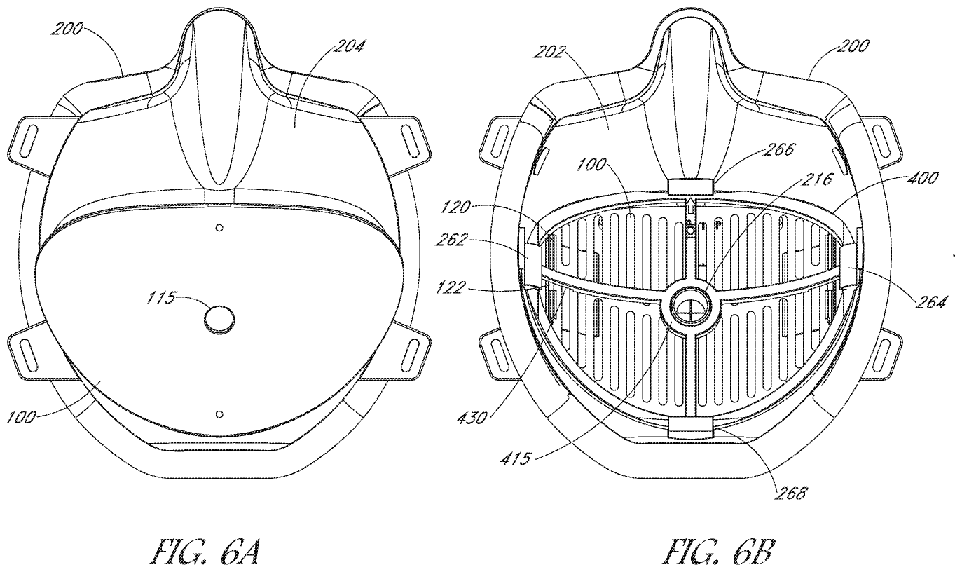

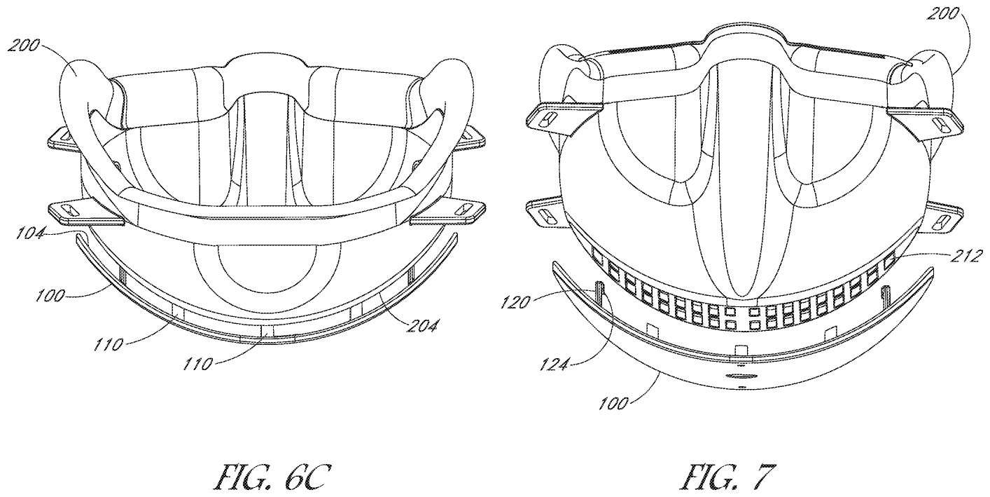

As shown in FIG. 6A, when the face plate 100 is attached to the front of the mask body 200, the face plate 100 advantageously overlies at least substantially the entirety of breathing area 210 of the mask body 200 when viewed from the front of the mask. Preferably, the width of face plate 100 may be somewhat greater than the width of breathing area 210, while the height of face plate 100 may be greater than the height of breathing area 210. By covering all, or at least substantially all, the breathing area 210 along a line of sight, the face plate 100 is disposed to block or obstruct the path of aerosol droplets exhaled through the breathing openings 212, while also blocking or obstructing the path of aerosol droplets that may be in the air around the wearer before they can enter the breathing openings. While aerosolized droplets small enough to be suspended in the air may still drift in and out of the breathing openings 212 via a fluid flight path around the sides of face plate 100, such aerosolized droplets are believed to carry fewer pathogenic particles than droplets that would travel along a direct flight path, and they are believed to have a higher likelihood of being stopped by a filter, or of being carried away by an errant breeze. Such a configuration also significantly reduces the burden of a filter that that may be used with the mask (as described below) to filter particles entering the interior of the mask body 200 from the outside, as fewer droplets have a direct flight path to the breathing openings 212 of the mask body 200. In other words, all aspects of filtration efficacy, such as bacterial filtration efficacy and particle filtration efficacy, are improved by obstructing the paths of particles and aerosols to and from the breathing openings 212 of the mask body 200.

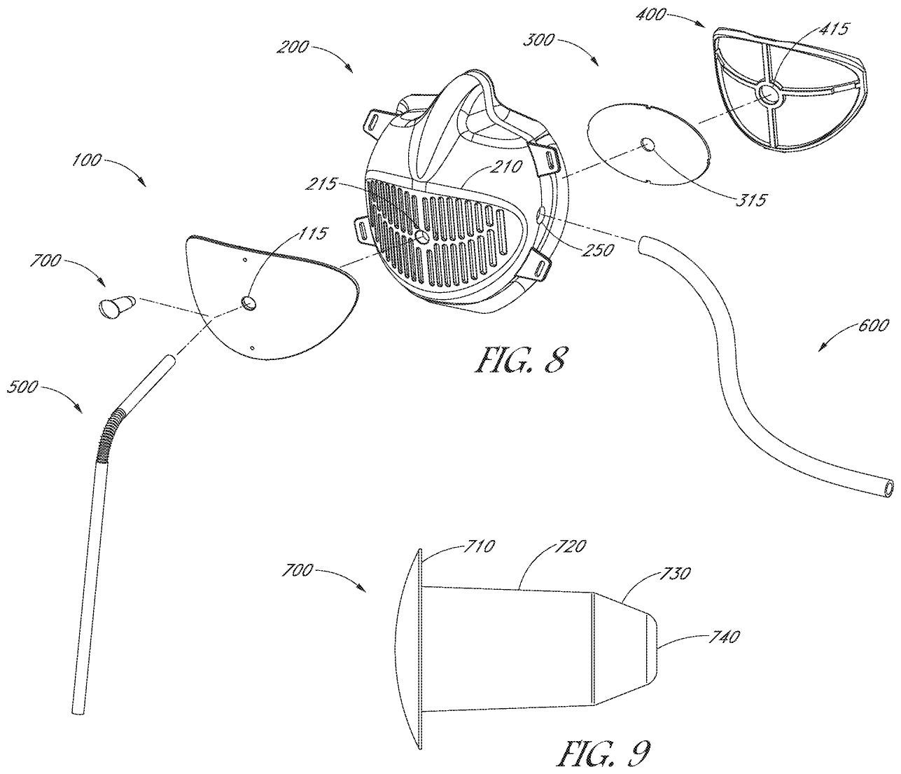

The face plate 100 may also have a plurality of standoffs 110 extending proximally from the rear surface 104. The standoffs function as spacers that engage the exterior surface 204 of the mask body 200 when the face plate 100 is attached thereto, as shown in FIGS. 6C and 7, so as to maintain the face plate 100 at a fixed distance from the breathing area 210, thereby allowing the passage of air around the edges of the face plate 100 and through the breathing openings or apertures 212 of the mask body 200 as the user inhales and exhales.

One manner of attaching the face plate 100 to the exterior or front of the mask body, in accordance with an aspect of this disclosure, is shown in FIG. 7. The hook portion 124 of each of the mounting elements 120 is configured to pass through an associated or corresponding attachment aperture in or near the breathing area 210 of the mask body. As shown in FIG. 7, each of the attachment apertures may be one of the breathing openings 212. While the mounting elements 120 are shown here as coupling to the outermost left and right breathing openings 212, other breathing openings 212, apertures or mounting configurations could be used. For example, in an alternative aspect, dedicated attachment apertures (not shown) may be provided adjacent to opposite edges of the breathing area 210. In either case, the face plate 100 may be flexibly manipulated to insert each of the hook portions 124 into its associated attachment aperture until the hook portion engages the interior surface of the mask body. The resilience of the face plate will provide a secure engagement of the face plate with the mask body. Detachment of the face plate 100 is performed by flexibly manipulating the face plate 100 to disengage the hook portions 124 from their respective attachment apertures. Other types of detachable mounting mechanisms may be used and will readily suggest themselves to those skilled in the art. For example, hook-and-loop fabric fasteners, snap-in fasteners, loop and post fasteners, and loop and button fasteners may be employed, and may be advantageous where a more rigid face plate is used.

The face plate 100 could be made from any suitable material, such as polypropylene or polycarbonate. In some aspects, a mask kit could provide a user with one mask body 200 and a plurality of interchangeable face plates, similar or identical to face plate 100, allowing for a user to apply different indicia labels to different face plates and change the look of the mask by interchangeably attaching different face plates to the front of the mask body 200. In some aspects, a mask kit could have one face plate that does not have an opening 115 and another face plate that does have an opening 115, allowing a user to alter the functionality of the mask, such as, for example, by blocking the straw port 215, simply by changing the face plate.

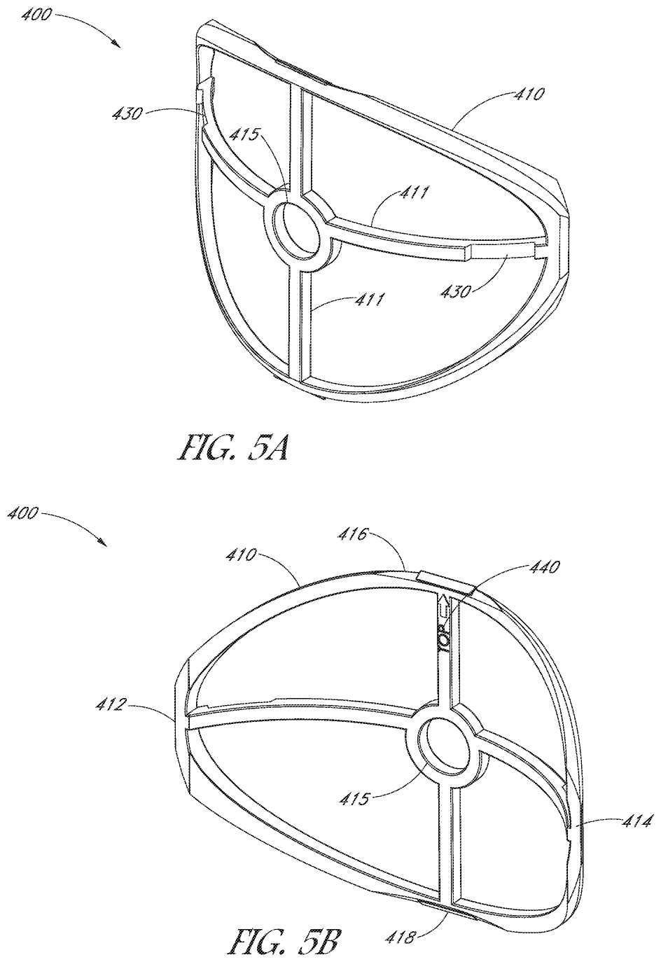

FIG. 4 illustrates a filter medium 300 that may be removably installed in the interior of mask body 200, in the manner described below. The filter medium 300 is sized to cover all or substantially all the breathing area 210 when installed at the interior surface 202 of the mask body 200. By covering the breathing openings 212 of the breathing area 210, the filter medium 300 provides filtration of sold particles and aerosol droplets in the air that passes through the breathing openings 212. The filter medium 300 may include a central opening 315 that is alignable with the straw port 215 of mask body 200. The filter medium 300 may be made of any suitable filtration material, such as filter paper (pleated or non-pleated) or a close-weave cloth, and it may include activated carbon and/or metallic (e.g., silver or copper) fibers or particles. The filter medium 300 may have physical demarcations, such as indents 310, that are configured to assure proper placement in a filter retainer element, as described below with reference to FIGS. 5A and 5B.

In some embodiments, portions of the filter medium 300 or portions of the mask body 200 may comprise an adhesive (not shown), or an adhesive may be added to either the filter medium 300 or the mask body 200, to allow the filter medium 300 to be removably coupled to the mask body 200 without use of the filter retainer element 400, or to hold the filter medium 300 in place against the mask body 200 before installing the filter retainer element 400. Such adhesives could be configured to be removable from the mask body 200 without damaging the surface of the mask body 200, for example adhesives that remain tacky without a hard set at room temperature.

FIGS. 5A and 5B illustrate a filter retainer element 400, in accordance with aspects of this disclosure, that may be removably installed in the interior of the mask body 200 to hold the filter medium 300 in place against the interior surface of the mask body adjacent the breathing area 210. The filter retainer element 400, in accordance with embodiments of the disclosure, is formed as an open frame defined by a perimeter member 410 and structural cross-members 411 that intersect with an annular central member 415 that may be provided to coincide with the central opening 315 of the filter medium 300 (FIG. 4) to accommodate a straw, should the wearer desire to use one.

The perimeter member 410 may have shaped filter retaining coupling surfaces 412, 414, 416, and 418 shown in FIG. 5B, that are configured to mate with matching mask body coupling surfaces 262, 264, 266, and 268, respectively, located on the interior of the mask body 200 shown in FIGS. 1B-1D. The matching coupling surfaces help a user to guide the filter retainer element 400 into a removable attachment state with the interior of the mask body, such that the filter medium 300 is secured adjacent the breathing openings 212. In some aspects, the retainer element 400 may be made of a material (e.g. polypropylene) that is somewhat more resilient than the material of the mask body (e.g. TPE), thereby allowing a snap fit between the coupling surfaces 262, 264, 266, and 268 of the mask body and the retainer element coupling surfaces 412, 414, 416, and 418. When a user presses such a resilient retainer element 400 towards the interior surface 202 of breathing area 210, each of the mask body coupling surfaces 262, 264, 266, and 268 could be configured to deform around the filter retaining coupling surfaces 412, 414, 416, and 418, respectively, to hold the filter retainer element 400 in place. In some aspects, the retainer element 400 may be made of a material that is somewhat more resilient than the material of the mask body, which could cause the filter retaining coupling surfaces 412, 414, 416, and 418 to deform away from the mask body coupling surfaces 262, 264, 266, and 268, respectively, during the coupling process. Any other suitable removable attachment mechanism could be used to install the filter retainer element 400 in the mask body 200, such as matching indents and detents, hook-and-loop fabric fasteners, snap fasteners, or a button clasp mechanism.

In some aspects, portions of the filter retainer element 400, such as the structural cross-members 411, may interfere with portions of the face plate 100, such as the mounting elements 120, when both the filter retainer element 400 and the face plate 100 are attached to the mask body 200 at the same time. To prevent the filter retainer element 400 and the face plate 100 from interfering with one another, notches could be formed in one or both of the elements to accommodate one another. In one aspect, the filter retainer element 400 may include notches 430 that are configured to provide space for the mounting elements 120 to rest when the face plate 100 is attached to the mask body 200, and the face plate 100 may include notches 122 that are configured to provide space for the structural cross-members 411 to rest when the filter retainer element 400 is attached to the mask body 200. The notches 430 could have a taper along each end which would help guide the corresponding notches 122 of mounting elements 120 to the right area. As a corollary, in embodiments where face plate 100 is attached to the mask body 200 before filter retainer element 400 is installed in the mask body 200, such tapers of the notches 430 would help filter retainer element 400 to self-align when being installed in the mask body 200. In another aspect of the invention, the structural cross-members 411 may not have any notches at all, and notches 122 could be formed deeper to better accommodate the size and shape of the structural cross-members 411, while in another aspect of the invention, the mounting elements 120 may not have any notches at all, and the notches 430 could be formed deeper to better accommodate the size and shape of the mounting elements 120. In still another aspect of the invention, both the structural cross-members 411 and the mounting elements 120 could be configured without notches, and may be positioned so as not to interfere with one another at all, for example in embodiments where the mask body has two or more sets of parallel bars forming breathing openings 212.

Alignment indicia 440 may be printed or embossed on a surface of filter retainer element 400 to provide a visual guide for proper alignment of the filter retainer element 400 when removably attaching the filter retainer element 400 to the mask body 200. Such alignment indicia are useful for filter retainer elements that are not symmetrical in a horizontal or vertical direction.

As shown in FIG. 6B, when the filter retainer element 400 is attached to the interior of the mask body 200, the coupling surfaces 262, 264, 266, and 268 of the mask body retain the coupling surfaces 412, 414, 416, and 418, respectively, of the filter retainer element 400 in place. Any filter medium, such as the filter medium 300, disposed between the filter retainer element 400 and the interior surface 202 of the mask body 200 would be held in place against the breathing openings 212 to filter air passing through the breathing openings 212. The structural cross-members 411 could apply pressure to the indents 310, causing the flexible filter medium 300 to slightly flex and fold around the structural cross-members 411 to help hold the filter medium 300 in place. The annular central member 415 is advantageously located so as to surround the collar 216 of the straw port 215, and may provide annular support to the collar 216, particularly in aspects where the annular central member 415 comprises a more rigid material than the collar 216. The notches 122 of the mounting elements 120 and the notches 430 of the filter retainer element 400 mutually receive one another to minimize movement of the filter retainer element 400 in the horizontal direction and movement of the face plate 100 in the vertical direction.

FIG. 8 illustrates an exploded mask assembly comprising a face plate 100, a mask body 200, a filter medium 300, and a filter retainer element 400. As shown, the openings in each of the mask elements about breathing area 210—the opening 115 in face plate 100, the straw port 215 in mask body 200, the opening 315 in the filter medium 300, and the opening 415 in the filter retainer element 400—are each aligned along an axis 510 to allow a straw, such as the straw 500, to pass through all of the openings to reach the mouth of a wearer. Such a configuration provides a series or “sandwich” of particulate barriers, in which the face plate 100 and the filter retainer element 400 may be considered the “outer” barriers, and the mask body 200 and the filter medium 300 may be considered the “inner” barriers. In some aspects, the materials used to construct the outer barriers may be more rigid than the materials used to construct the inner barriers to help reinforce the path taken by the straw 500. For example, in some aspects, the face plate 100 and the filter retainer element 400 may both comprise a polypropylene material, while the mask body 200 may comprises a TPE material, and the filter medium 300 may comprise a cloth, fabric, or paper filter.

The sipping tube 600 is shown extended through the sipping tube port 250 to allow a wearer to sip on liquids. In some aspects, the collar 252 of the sipping tube port 250 may a greater distance between its inner and outer perimeters than the collar 216 of straw port 215, if, for example, the sipping tube 600 is thicker and sturdier than straw 500, and hence requires more support. The sipping tube 600 could be connected to or be a part of an external beverage source, such as a water backpack water pouch (not shown), or a straw that leads to a canteen (not shown).

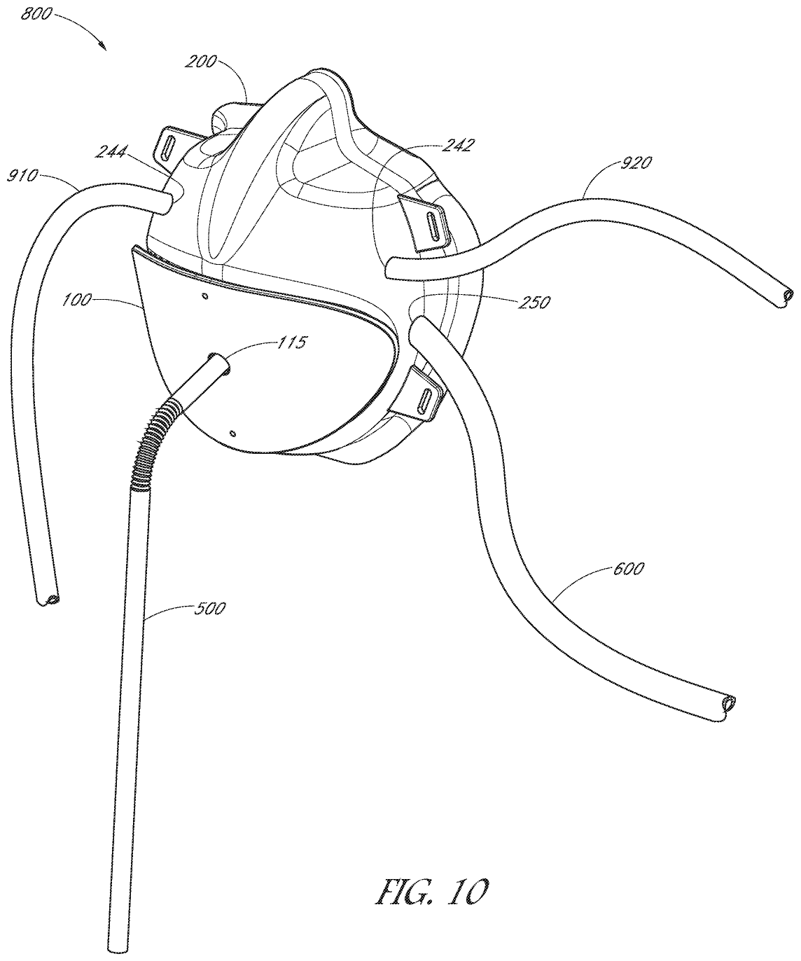

FIG. 9 illustrates an exemplary embodiment of a plug or closure 700 that may optionally be used to close an opening, such as any previously-opened port in the mask body. In the illustrated embodiment, the plug includes a head 710, a cylindrical body portion 720, a tapered body portion 730, and an end 740. In some aspects, a mask kit could provide a plurality of plugs, each of which is specifically adapted to plug a port that has been opened, but a user later wishes to close when not in use. For example, the plug 700 could be adapted to plug either the opening 115 of the face plate 100 or the straw port 215 of mask body 200. The plug 700 could be made from any material, such as an elastic material like TPE or rubber.

FIG. 10 illustrates an assembled mask assembly 800 showing a face plate 100 removably attached to a mask body 200. Each of the ports of the mask assembly 200 is in use, whereby a straw 500 is inserted through the face plate 100 and the mask body 200 via the opening 115 of the face plate 100; a sipping tube 600 is inserted through the mask body 200 via the sipping tube port 250; and oxygen tubes 910 and 920 are respectively inserted through the left oxygen port 242 and the right oxygen port 244 of the mask body 200. A wearer of the mask assembly 800 could seal any of the open ports using a plug, e.g., the plug 700, and could change the face plate 100 with another face plate to present a different indicia label.

Although various aspects of a mask assembly are shown, the components, and related methods have been specifically described and illustrated herein, and many modifications and variations will be apparent to those skilled in the art. Furthermore, it is understood and contemplated that features specifically discussed for a mask apparatus in accordance with any one aspect or embodiment of this disclosure may be adopted for inclusion with other such mask aspects, provided the functions are compatible. Accordingly, it is to be understood that the disclosed mask apparatus embodiments, their components, and related methods according to this disclosure may be embodied other than as specifically described herein.

What is claimed is:

1. A mask assembly comprising:
a mask body configured to cover a nose and mouth of a wearer, wherein the mask body defines an exterior surface and an interior surface and has a breathing area having breathing openings to permit passage of air between the exterior surface and the interior surface;
a face plate removably attachable to the front surface of the mask body, wherein the face plate, when attached to the front surface of the mask body, is configured to cover a line of sight of at least some of the breathing openings, and the face plate having a rear surface that is spaced from the breathing openings; and
a filter medium detachably fixed to the interior surface of the mask body so as to cover the breathing openings.

2. The mask assembly of claim 1, wherein the face plate has a front surface, and wherein the mask further comprises an indicia label on the front surface of the face plate.

3. The mask assembly of claim 1, wherein the face plate comprises a plurality of standoffs extending from the rear surface of the face plate that are removably attachable to the exterior surface of the mask body.

4. The mask assembly of claim 1, wherein the mask body comprises a straw port in the breathing area.

5. The mask assembly of claim 4, wherein the straw port comprises a pierceable element surrounded by a reinforcement collar.

6. The mask assembly of claim 4, wherein the face plate includes an opening that aligns with the straw port when the face plate is attached to the exterior surface of the mask body.

7. The mask assembly of claim 1, further comprising a filter retainer element removably attachable to the interior surface of the mask body and configured to hold the filter medium in place against the breathing openings.

8. The mask assembly of claim 6, further comprising a filter retainer element removably attachable to the interior surface of the mask body and configured to hold the filter medium against the interior surface of the mask body;
wherein the filter medium has an opening;
wherein the filter retainer element has an opening; and
wherein the opening of the filter medium, the opening of the filter retainer element, and the straw port are alignable with one another when the filter retainer element is attached to the interior surface of the mask body with the filter medium.

9. The mask assembly of claim 1, wherein the mask body comprises at least one tube port outside the breathing area.

10. The mask assembly of claim 1, wherein the mask body comprises a first straw port located within the breathing area, and a second straw port located outside the breathing area.

11. A hygienic mask assembly, comprising:
a mask body configured to cover a nose and mouth of a wearer, wherein the mask body comprises an exterior surface and an interior surface and has a plurality of breathing openings to permit passage of air between the exterior surface and the interior surface;
a face plate removably attachable to the exterior surface of the mask body so as to be spaced therefrom so as to permit passage of air through the breathing openings; and
a filter medium removably installable in the interior surface of the mask body to cover at least most of the breathing openings.

12. The hygienic mask assembly of claim 11, wherein the face plate has a front surface and a rear surface, the face plate further including a plurality of standoffs extending from the rear surface of the face plate to abut the exterior surface of the mask body.

13. The hygienic mask assembly of claim 11, wherein the mask body comprises a flange configured to obstruct passage of air between the exterior surface and the interior surface when the mask is worn by the wearer.

14. The hygienic mask assembly of claim 13, further comprising a first head strap and a second head strap, wherein the mask body comprises a pair of upper eyelets extending from opposing sides of the mask body and a pair of lower eyelets extending from opposing sides of the mask body below the pair of upper eyelets.

15. The hygienic mask assembly of claim 14, wherein the first head strap, when passed through the pair of upper eyelets, is configured to allow tightening of the engagement of the flange against the nose of the wearer when the mask body is worn on the face of the wearer.

16. A hygienic mask kit, comprising:
a mask body configured to cover a nose and mouth of a wearer, wherein the mask body comprises a breathing area comprising (1) a plurality of breathing openings between an interior surface of the mask body and an exterior surface of the mask body, and (2) a pierceable straw port;
a face plate removably attachable to the exterior surface of the mask body so as to be spaced from the exterior surface of the mask body, wherein the face plate is configured to overly the breathing area, and wherein the face plate comprises a face plate opening; and
a filter medium removably installable on the interior surface of the mask body to cover at least most of the breathing openings, wherein the filter medium comprises a filter medium opening, and wherein the filter medium opening, the face plate opening, and the straw port are alignable with one another when the filter medium is installed on the interior surface of the mask body and the face plate is attached to the exterior surface of the mask body.

17. The hygienic mask kit of claim 16, further comprising a filter retainer element removably attachable to the interior surface of the mask body to hold the filter medium in place against the interior surface of the mask body, wherein the filter retainer element has a filter retainer opening, and wherein the filter retainer opening, the filter medium opening, the face plate opening, and the straw port are alignable with one another when the filter retainer element is attached to the interior surface of the mask body with the filter medium.

18. The hygienic mask kit of claim 16, wherein the straw port comprises a pierceable element surrounded by a reinforcement collar.

19. The hygienic mask kit of claim 16, further comprising a plug configured to fit into and seal at least one of the straw port and the face plate opening.

20. The hygienic mask kit of claim 16, further comprising a first head strap and a second head strap;
wherein the mask body comprises a pair of upper eyelets extending from opposing sides of the mask body and a pair of lower eyelets extending from opposing sides of the mask body below the pair of upper eyelets;
wherein the first head scrap is configured to be passed through the pair of upper eyelets and to pass through one of the pair of upper eyelets and with one of the pair of lower eyelets; and
wherein the second head strap is configured to pass through the pair of lower eyelets and to pass through one of the pair of upper eyelets and with one of the pair of lower eyelets.
